(19) United States
(12) Patent Application Publication
(10) Pub. No.: US 2026/0041374 A1
(43) Pub. Date: Feb. 12, 2026

(54) PLATFORM FOR MOUNTING DEVICES INSIDE SCANNER BORES

(71) Applicant: Medical Devices Corner, Inc., Menlo Park, CA (US)

(72) Inventors: Samuel Frishman, Menlo Park, CA (US); Tianyang Lei, Menlo Park, CA (US)

(21) Appl. No.: 19/122,295

(22) PCT Filed: Oct. 18, 2023
(86) PCT No.: PCT/US2023/077211

§ 371 Date: Apr. 18, 2025

Related U.S. Application Data
(60) Provisional application No. 63/417,112, filed on Oct. 18, 2022.

(51) Int. Cl. A61B 5/00; A61B 5/055

(52) U.S. Cl. CPC A61B 5/704; A61B 5/055

(57) ABSTRACT

A system for use in a medical scanning device that includes a base fixture; an in-bore fixture with a semi-circular outer profile, and which comprises adaptive borewall pressure loads distributed across multiple locations on the outer profile, wherein the adaptive borewall pressure loads direct pressure radially outward; and a boom support attachable between the base fixture and the in-bore fixture.

### CROSS-REFERENCE TO RELATED APPLICATIONS

This Application is a National Stage Application under 35 U.S.C. 371 which claims the benefit of PCT Patent Application No. PCT/US2023/077211, filed on 18 Oct. 2023, which claims the benefit of claims the benefit of U.S. Provisional Application No. 63/417,112, filed on 18 Oct. 2022, both of which are incorporated in their entirety by this reference.

### TECHNICAL FIELD

This invention relates generally to the field of medical scanners, and more specifically to a new and useful system for a platform used for mounting devices within a scanner bore.

### BACKGROUND OF THE INVENTION

MRI (Magnetic Resonance Imaging) machines have proven incredibly useful in the medical field. Traditionally, MRI machines are used for imaging of a patient. While the visual data can be incredibly useful to doctors, caretakers, and/or researchers, MRI machines are accompanied by various limitations that have prevented them from being used in some circumstances. For example, the type of objects that can be introduced inside and/or near the MRI machine is limited. Metal objects, for example, cannot be used when performing MRI imaging.

Additionally, it can be challenging to introduce other objects into the bore of an MRI machine for use while a patient is being imaged. The space within the bore of an MRI machine does not leave much room when a patient is inside. Additionally, most MRI machines and other scanners fail to provide mechanism to which to mount or integrate additional equipment.

As another challenge, the availability of the machines can often be very constrained. It can be desirable for patients to be efficiently situated into a scanner machine and the imaging procedure performed. Any operational complexities for introducing objects and systems within a device can face logistical challenges because of the practical efficiency requirements. Any existing solution may be too slow and cumbersome to be a practical solution in the medical field.

As another challenge, ferrous metal and other materials cannot be used within the bore of an MRI, and so there are practical mechanical challenges of how to actual situate any objects inside the MRI machine.

As another challenge, any improvements to the design of MRI machines will take decades to become widely available. MRI machines are extremely expensive devices, and their lifetime is very long, and so even if some newly designed MRI machine were to be introduced there would still be many existing machines that still experience the limitations described herein.

Thus, there is a need in the medical scanner field to create a new and useful system and method for a platform used for mounting devices within a scanner bore. This invention provides such a new and useful system and method.

### BRIEF DESCRIPTION OF DRAWINGS

FIG. 1 is a schematic representation of a system variation.

FIG. 2 is a schematic representation of a system variation.

FIG. 3 is a schematic representation of a system in two possible states.

FIG. 4 is a detailed schematic representation of a sled attachment fixture.

FIG. 5 is a detailed schematic representation of a system from a view aligned with a longitudinal axis.

FIG. 6 is a detailed schematic representation of one in-bore fixture variation.

FIG. 7 is a detailed schematic representation of a spring-loaded wheel.

FIGS. 8-10 are variations of adaptive borewall pressure loads.

FIG. 11 is a schematic representation highlighting an attachment between the in-bore fixture and a boom support.

FIG. 12 is a schematic representation of an articulating mounting fixture.

FIG. 13 is a schematic representation of an in-bore fixture variation without ground contact.

### DETAILED DESCRIPTION OF THE EMBODIMENTS

The following description of the embodiments of the invention is not intended to limit the invention to these embodiments but rather to enable a person skilled in the art to make and use this invention.

### 1. Overview

A system for a platform used for mounting devices within a scanner bore can function as an adaptable structure that accounts for various technical and usability constraints. The system is preferably used as a platform for mounting scanner-compatible objects inside a scanner bore of an MRI (Magnetic Resonance Imaging) machine or a CT (Computerized Tomography) scanner, though it may be used in orienting or positioning inside any suitable type of device. In particular, the system can provide a mechanical robust mounting structure, suitable for use with mounted medical equipment, while being made fully of material compatible with an MRI device or other type of scanning device.

In one variation, the system can use two locations of fixturing that leverage ground reaction forces to achieve a stiff mounting structure. A first fixturing location can be at the front of a scanner sled (e.g., at end of the scanner sled furthest from the scanner bore). A second fixturing location can be positioned near the back of the scanner sled. The second fixturing location may additionally engage with the bore wall using a curved structure (e.g., a semi-circle structure) with adaptive pressure loaded elements (e.g., pads or wheels) to engage with the bore wall. When used, a patient may lay between the two fixturing locations. A detachable or adjustable boom may be mounted between the two fixturing locations. Objects can be mounted to one or more locations of the platform of the system. The system can include various positioning degrees of freedom to translate/rotate an object into a desired position.

The system can be used to enable custom positioning of one or more objects within the bore. In some variations, the system can enable custom positioning with controlled longitudinal position, radial displacement, and/or angular position. In some alternative variations, the system may enable mounting with orientations that vary in other ways.

Using a cylindrical coordinate system, a defined longitudinal axis will be characterized as an axis extending through the defined cavity of the bore and being substantially centrally located. Longitudinal position will be used to characterize the position along the longitudinal axis. Radial displacement will be used to characterize the distance extending perpendicular from the longitudinal axis. Angular position will be used to characterize the angle within a defined plane perpendicular to the longitudinal axis. Reference to lateral axis, lateral direction, or width may refer to the side-to-side directions (width wise), and the lateral axis is perpendicular to the longitudinal axis.

The system can be used to position any suitable type of object and/or number of objects. In one particular application, the system can be used for mounting a medical device that performs some action on the patient. For example, a scanner-compliant surgical tool could be mounted to the system and used to perform an operation on a patient while the patient is being imaged by the scanner. In another example, a static object or objects may be mounted in proximity to a patient, which may be used by a patient during a scan or otherwise. Additionally, the system may serve as a platform for multiple distinct objects.

The components of the system may be designed using materials and sub-components that are compatible with the targeted type of scanner. For example, the components of the system may be made of non-metal materials. For example, all the components may be made of a polymer or materials compatible with MRI devices such as polycarbonate, acrylic, ceramic, or other suitable materials. The components could similarly be made of materials satisfying other requirements such as being: non-magnetic materials, non-conductive materials, radio frequency (RF) transparent materials, non-ferromagnetic materials, and/or other material types that would be suitable for use in restrictive scenarios. This may make the systems and methods compatible for use alongside MRI (Magnetic Resonance Imaging) devices, CT (Computed Tomography) devices, and/or other sensitive devices. Additionally, the systems and methods may not require use of active components within the physical structure. For example, the systems and methods may not have any motors and electronics. While the system may be particularly suited for use within a device with a large bore that has restricted material usage, the system may alternatively be made of any suitable material and used in other applications.

The system and method may provide a number of potential benefits. The system and method are not limited to always providing such benefits and are presented only as exemplary representations for how the system and method may be put to use. The list of benefits is not intended to be exhaustive and other benefits may additionally or alternatively exist.

As one potential benefit, the system can provide a flexible mounting platform for a variety of applications. Most scanner devices do not include any design features to enable easy positioning an object around a patient. The system can be adjusted in many ways to enable many mounting options.

As a related benefit, the system may also be a substantially stable mounting platform. The system can use a mounting approach that engages with a scanner bore wall so as to efficiently restrict motion. The system can provide a highly secure and rigid mounting structure that mitigates movement or vibrational motion relative to the patient. This can be important when the mounted devices depend on a stable positioning. For example, the system may be used for mounting a surgical medical device that operates on the patient while in the scanner.

As another benefit, the system can also be used without complicating the use of the scanner by a physician and/or patient. The system can be quickly configured to remove components that would otherwise restrict motion of a patient when the patient is getting onto or off a scanner sled. For example, the boom arm may be detached from one or more of the fixturing points and then rotated or otherwise moved out of the way. Similarly, the system can allow the mounting platform to be quickly removed from the scanning device or added to a scanning device for efficient setup and/or breakdown.

As another benefit, the system may be usable in a variety of types of legacy scanning devices. In this way, the system can serve as a retrofit to existing scanner devices to enable new in-bore mounting options. This retrofitting of scanner device, furthermore, can be done without physically altering the scanner devices. This can be important because such scanner devices can be incredibly expensive, and hospitals may be resistant to permanently altering such a device. The mounting solution of the system may be a more desirable option because it may not require permanent changes to the expensive and existing scanning device.

As another benefit, the system can offer the mounting solution using physical elements made from scanner-compliant materials. For example, the physical structures of the system may be made of polymer-based materials like polycarbonate or other types of materials. This can enable the system to be used during use of the scanner device.

### 2. System

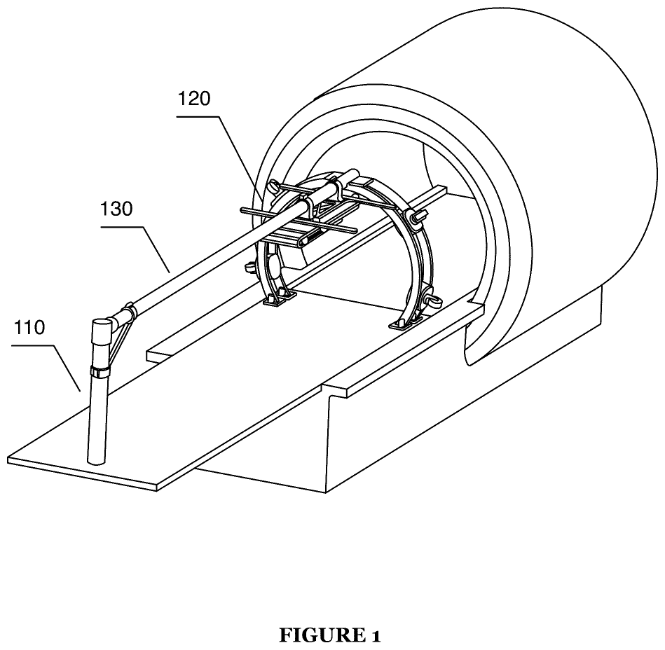

As shown in FIG. 1, a system for mounting devices inside a scanner bore can include a base fixture 110, an in-bore fixture 120, and a boom support 130 attachable between the base fixture 110 and the in-bore fixture 120. The system can additionally include one or more mounting fixtures 140 that is part of or connects to one of the system components to facilitate mounting one or more devices.

Some variations, use a borewall pressure loads 122 along an outer perimeter to engage with a borewall of a scanning device. Accordingly, the system of some variations may include a base fixture 110 attached to a front end of a sled of a scanner device; an in-bore fixture 120 with a semi-circular outer profile and that includes adaptive borewall pressure loads 122 distributed across multiple locations on an outer profile of the in-bore fixture, where the adaptative borewall pressure loads 122 direct pressure radially outward; and a boom support 130 that is attachable between the base fixture 110 and the in-bore fixture 120. The boom support 130 and/or the in-bore fixture 120 may be used to provide one or more mounting fixtures 140 to which one or more devices can be attached and oriented.

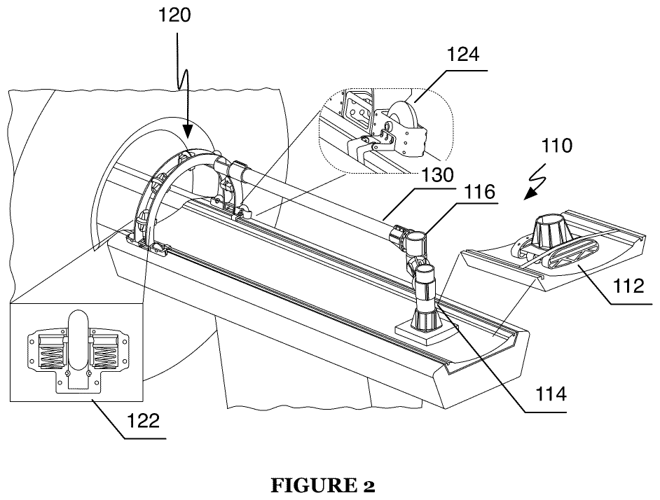

In some variations, as shown in FIGS. 1 and 2 the base fixture 110 can include a sled attachment fixture 112 that interfaces or contacts with a sled of a scanning device, a vertical structure 114, a boom attachment mechanism 116 to connect to the boom support, and optionally an actuating connection with the boom support. The in-bore fixture 120 can have a semi-circular outer profile and in some variations may be a semi-circular partial ring structure. The in-bore fixture 120 includes a structural frame, a set of pressure loads distributed across multiple locations on the semi-circular outer profile of the in-bore fixture. The semi-circular partial ring structure may terminate at two grounding contacts 124 on opposing sides of the sled. The system can additionally include one or more types of mounting fixtures that facilitate adapting the system as a mounting platform for connected devices.

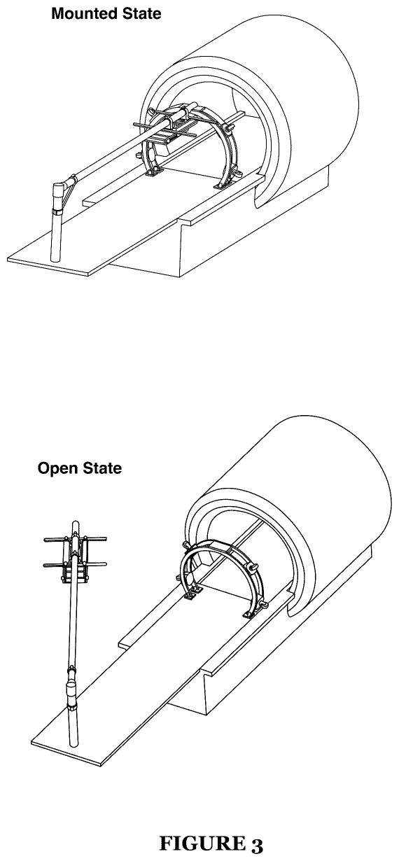

The system can preferably be reconfigurable between a mounted state and an opened state such as shown in the exemplary variation of FIG. 3. In the mounted state, the system is rigidly engaged with the scanning device. The mounted state is usually used while the patient is positioned within the scanner. There may be a partially mounted state and a fully mounted state. The partially mounted state may be when the boom support 130 is connected to the base fixture 110 and the in-bore fixture 120, but the adaptive borewall pressure load 122 is not fully engaged. The fully mounted state may be when the adaptive borewall pressure load 122 is engaged with the bore wall of the scanner. In the opened state, the system can be configured to leave the sled more open so that a patient (or subject) can get onto or off the sled.

Herein, the system is described as using one base fixture 110 and an in-bore fixture 120. Alternative configurations may alternatively be used. In some alternative variations, the system may use a single in-bore fixture 120, which may or may not include a boom support 130 or a base fixture 110. The system could alternatively include two or more in-bore fixtures 120, which may also include a boom support 130 that can be suspended between the two or more in-bore fixtures 120.

The system is additionally described in a configuration with the base fixture 110 positioned at the front of a sled and the in-bore fixture 120 positioned at the back of the sled. However, the system may alternatively use alternative positioning while retaining the functionality and core components of the system. For example, the base fixture 110 may be positioned at the front of the sled, and the in-bore fixture 120 may be positioned in the middle region of the sled, and a boom support 130 attached to the base fixture 110 and the in-bore fixture 120 can extend beyond the in-bore fixture 120 to extend to the back region of the bore if necessary.

The system is preferably used with a scanner device like an MRI imaging device or a CT scanner. The system may be used in other situations, especially those where a device mounting solution may be desirable within a small, constrained space. The system may be particularly useful in applications where there is a defined cylindrical cavity (e.g., a bore of some device) and a platform or surface (e.g., a sled) that slides, or which can have a slidable platform added. Herein, references to MRI imaging devices are used for sake of convenience and clarity of examples, but the system is not limited to use with just MRI devices.

In general, the system is described as a system that may be used to augment an existing scanner. The system may be a standalone system that can be introduced and used with an existing MRI device, for example. In some alternative variations, the system may include the scanner device. A scanner device may be designed with physical attributes to directly accommodate such a mounting system. For example, a device's sled may have a base fixture 110 directly integrated into it.

The scanner will generally include a defined inner bore cavity that is referred to herein as the bore. The bore will generally be a substantially cylindrical in shape but may alternatively have any suitable shape profile. In many actual uses, the bore of a MRI device is not a perfect cylindrical shape but may be a cavity with a shape profile that includes an at least a portion that is a semi-circular or rounded in profile. For example, the bore in some devices may be a cavity defined as an extruded semicircular shape. The surface of the scanner surrounding a defined bore cavity is referred herein as a bore wall. Herein, references to “in-bore” are used to characterize being intended positioning in the volume of the defined bore cavity. In the case of the in-bore fixture 120, the fixture may be external to the bore during some instances, but during active use, the in-bore fixture 120 will generally be contained within the bore. The bore wall in some scanner variations may be made of a substantially continuous surface such as a plastic sheet formed into a cylindrical or rounded shape.

The scanner will generally include a sled (or bed). The sled can be the platform on which a subject (e.g., a patient) is positioned during use of the scanner. The sled in some variations is movable so that it can slide in and out of the bore of the scanner. The sled may additionally rise and lower. A patient will generally lie down on the sled while it is slid out, and then the sled is moved to position the patient (or a portion of interest of the patient) into the bore. The sled may be described as having a front end (e.g., distal end) that is characterized as the end furthest away from the scanner when positioned outside of the scanner. The sled may also be described as having a back end (e.g., proximal end) that is characterized as the side of the sled closest to the scanner (e.g., the first end to move into the bore). A patient in many cases will have their head positioned nearer to the back end of the sled and their feet nearer to the front end of the sled.

The base fixture 110 functions to provide a rigid mounting structure on one end of the sled. The base fixture 110 may be a rigid structure that is intended to extend up above the sled such that it can support a boom resting at an elevated position. The height where the base fixture 110 supports the boom is preferably high enough such that the boom can pass over the patient subject when the patient is resting on the sled.

The base fixture 110 may be used to restrict position of at least one end of the boom support 130. In particular, the base fixture 110 may be used to fix motion of the boom support 130 axially (along the defined longitudinal axis in/out of the bore) and height of boom on one end. In other words, the base fixture 110, when positioned and fixed to a sled (at least temporarily), can preferably not move longitudinally along the sled. The base fixture 110 may also be fixed side-to-side. The base fixture 110 can additionally include substantially rigid components used to achieve the height to support the boom support 130. The rigid components may extend from a base surface where the base fixture 110 contacts the sled to where the base fixture 120 connects to the boom support 130, thereby restricting vertical displacement at least on that end of the boom support 130. When the boom support 130 is mounted, then the in-bore fixture 120 may correspondingly be substantially limited in longitudinal movement.

The base fixture 110 may include a sled attachment fixture 112 that is used to engage with some bottom support (e.g., the surface of the sled). The base fixture 110 can additionally include a vertical structure 114 extending from the sled attachment fixture 112 upwards. The vertical structure 114 functions to achieve a desired mounting position for where the boom support 130 will attach.

The base fixture 110 can additionally include a boom attachment mechanism 116 that functions to connect the base fixture 110 to the boom support 130. In one variation, the boom attachment mechanism 116 may be a multi-segmented arm with adjustable degrees of freedom. This may allow for adjustable positioning of where the boom support 130 is positioned.

The base fixture 110 may additionally include an integrated angular degree of freedom, which functions to allow swinging the boom support 130 into an open position as shown in FIG. 3. The base fixture 110 may include a rotational joint (or hinge or form actuation mechanism) and a boom attachment mechanism 116 to selectively couple the boom structure with the in-bore fixture. The boom arm may be rotated along an angular degree of freedom to move the boom structure to engage and disengage with the in-bore fixture 120.

The angular degree of freedom is preferably about the vertical axis. However, some alternative variations may set the axis of rotation differently. For example, an alternative joint may be oriented to cause the boom support 130 to swing out and up. Another alternative joint may be oriented to cause the boom support 130 to swing out and down.

In one variation, the vertical structure 114 may have an integrated joint so that the upper portion of the vertical structure 114 can rotate. If the boom support 130 is attached to the vertical structure 114, then this will cause the boom support 130 to similarly rotate.

In another variation, the boom attachment mechanism 116 may include a joint or hinged connection to the boom support 130. For example, the connection between the boom support 130 and the base fixture 110 may itself be a joint. The joint can function to enable the boom support to be rotated out.

In one variation, the base fixture 110 may support mounting the boom support 130 at variable heights. In one variation, the vertical structure 114 may be extendable such that the height of the vertical structure 114 can be adjusted using some sliding mechanism, telescoping mechanism, or any suitable mechanism to make the fixed height of the vertical structure 114 adjustable. In another variation, the boom attachment mechanism 116 may support the boom support 130 being attached at a range of different heights. For example, the boom attachment mechanism 116 could have a collar design, where it can be adjusted to a desired heigh along a cylindrical vertical structure 114 and then tightened or otherwise fixed in place.

The base fixture 110 in one variation is mounted and physically coupled to the sled, more specifically to the top surface of the sled. In some variations, the base fixture 110 may be attached in a way that it can be easily added and removed from the sled. As an easily attachment component, the base fixture 110 and the system as a whole may be more easily assembled and dissembled within a shared device. In other variations, the base fixture 110 may be attached to the sled in a permanent or semi-permanent manner. In these variations, the sled (or scanner device more generally) may integrate attachment mechanisms or a base fixture 110 component directly.

The base fixture 110 is preferably mounted at the base of the sled as discussed above. The base fixture 110 is preferably mounted in a central location along the width of the sled. A central mounting position may enable a patient's feet/legs to straddle the base fixture 110 if needed. The base fixture 110 is not limited to being mounted at or near the base of the sled and/or in a central position laterally. For example, an alternative variation of the base fixture 110 may be designed to be mounted to one side. Optionally, for such a side-mounted base fixture 110, an arm structure could extend to a central point for mounting the boom support 130 if a centrally positioned boom support 130 is desired.

The base fixture is preferably fixed into a static position. In particular the base fixture may be fixed in position on the sled of a device. The base fixture 110 may be positioned using a variety of approaches. The base fixture 110 may include a sled attachment fixture 112 that can include structural features used to facilitate mounting or attaching to a sled.

In one variation, an adhesive such as dry adhesive may be used to adhere the base fixture 110 to a surface of the sled. Accordingly, the base fixture may include a bottom surface that is adhered to a surface of a sled. Dry adhesive may be used to glue the bottom of the base fixture 110 to a sled. In some variations, the sled attachment fixture 112 of the base fixture 110 may be a structure with a flat surface or a surface that can conform to the surface of a sled. The sled attachment fixture 112 may flair out to increase surface area contact with a sled. A dry adhesive or other type of temporary or permanent adhesive to adhere to the sled attachment fixture 112.

In another variation, the base fixture 110 is rigidly attached to the sled semi-permanently (or permanently). This may include attaching via bolts, welding, or other permanent or semi-permanent approaches. In these variations, the sled may be designed with the base fixture 110 as an integrated component or feature. Alternatively, a sled may be designed specifically with an adapter to attach to the base fixture 110.

In another variation, a separate attachment mechanism may be used to attach the base fixture 110 to the sled. The system may include an attachment connector that physically couples a sled and a sled attachment fixture 112. The attachment connector could include latches, clamps, or other fixturing mechanisms that can be used to fix the bottom of the base fixture 110 to a location on the sled. The attachment connector can attach to a part of the sled and/or to the sled to restrain position of the base fixture 10. In one variation, the sled may include grooves along either side, that are traditionally used for restraint straps to secure a patient on a sled. The sled attachment fixture 112 may include clamps or another locking mechanism that can engage with the grooves and restrict motion. In some variations, once engaged, the attachment connector may be tightened or otherwise locked to fix position.

In related variation, the attachment mechanism may be adapted to have straps restrict its position. Existing straps or grooves or other attachment for straps may be used to restrict motion of the base fixture 110. In one variation, two or more straps may wrap over either side of the sled attachment fixture 112. Tightening the straps can preferably provide a downward force on the sled attachment fixture 112. A strap on either side of a central vertical arm can further restrict motion longitudinally. The sled attachment fixture 112 may include various design features to enhance security of such a fixturing approach.

In one variation, the base fixture includes a sled attachment fixture 112 that is strapped to a sled of the device. One or more straps can be wrapped across or otherwise engage with the sled attachment fixture 112. When the straps are tightened, the sled attachment fixture 112 may be configured with the arrangement of the straps to apply restrictive forces to restrict motion of the base fixture 110. In particular, the sled attachment fixture 112 can be configured to achieve downward forces when straps are wrapped and tightened.

In one strapping approach, straps are wrapped from one side to an opposing side. In another strapping approach, multiple straps may be wrapped in an overlapping pattern. In another strapping approach, multiple straps may extend from the sled to engage with some point on the sled attachment fixture 112 and then tighten. In this way multiple lengths of straps can be tightened. When multiple lengths of straps.

Herein, strap is used generally refer to any member that can extend from a connecting point on the sled to the sled attachment fixture 112. The trap may be a flexible fiber strap, a cord, or a wire. A rigid link with latching mechanisms may similarly be used. The rigid link may be wrapped around the sled attachment fixture 112 in place of a strap, and then latched or engaged with the sled and/or sled attachment fixture 112.

In one variation, the sled attachment fixture 112 includes a convex bottom surface. Many sleds will be concave, and so a convex bottom may facilitate better pairing of the bottom surface of the base fixture 110 to the top surface of a sled. Alternatively, the bottom surface of the sled attachment fixture 112 may include a flat surface or any suitable surface structure.

In one variation, the sled attachment fixture 112 includes a conformant bottom surface, which functions as deformable surface to adapt the bottom surface of the base fixture 110 to the sled surface. This may make the base fixture 110 adaptable to a variety of sled. Different MRI machines may have different sled designs. Some sleds may be flat while others may have various contoured profiles.

In particular, the sled attachment fixture 112 includes conforming material along a bottom surface. A conforming material may deform such that contact can be made across a wider surface area of the sled regardless of exact contours. The conforming material may be made of a solid flexible material. Alternatively, the conforming material may include a structural lattice that allows the bottom surface to adjust while remaining structurally secure when strapped down. In some variations, the conformant bottom surface may also be a convex bottom surface.

The sled attachment fixture 112 may additionally or alternatively include a bottom surface that is a grooved surface and/or be made of a high friction material. High friction materials may include materials such as rubber, silicones, urethanes, textured or roughened rigid materials (e.g., textured metals or plastics), cork, and/or other materials. Such features may increase friction forces when in contact with the surface of the sled. The materials may be selected to be compatible with the intended use such as MRI compatible materials.

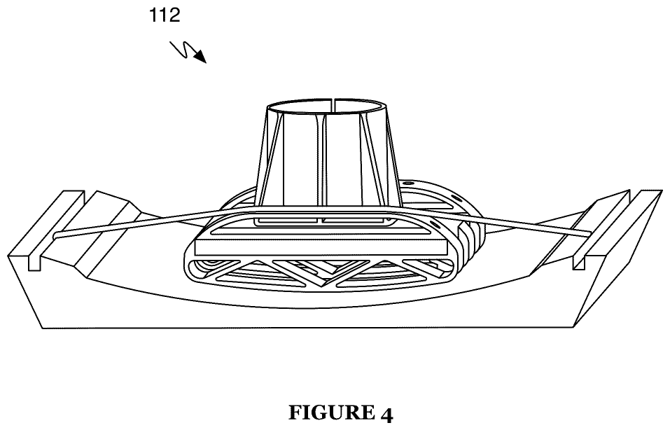

A sled attachment fixture 112 can additionally include at least one structural element that extends vertically to a height above where a strap attaches to a sled. The straps may be attached within a groove on either side. They may be at a height greater than the lowest height of the sled surface as in the example shown in FIG. 4. Such a structural element may be referred to as an elevated structure. The straps will preferably be wrapped over the elevated structure. When tightened, the elevated structure will result in more of a downward force on the base fixture 110. The elevated structure may have a single point elevated, which will cause the straps to rise up and possible change direction about the point. The elevated structure may alternatively have an extended surface at an elevated height over which straps may run. In some variations, the elevated structure may similarly include features to increase friction or to better grip the straps to avoid strap slippage. The elevated structure may include grooves or other surface patterns and/or made of high friction material.

In one variation, the sled attachment fixture 112 may include strap guide structures. The strap guide structures function to restrict and/or guide a strap that is used to restrict motion of the base fixture 110. A strap guide structure may be a defined through-hole or slit through which a strap may be passed. The strap guide may alternatively be a recessed path or guide structures (e.g., a raised overhanging tab) that restricts a strap to a designated region. The strap guide may prevent a tightened strap from slipping off. There can be multiple strap guides for a single strap or for multiple straps.

In a related alternative or similar variation, the sled attachment fixture 112 may include a structural element that both serves as an elevated structure and as a strap guide.

The sled attachment fixture 112 may additionally or alternatively include other features to better engage with or adapt to the sled surface.

In some variations, the sled attachment fixture 112 may be interchangeable. The system can include sled adapters, where a given sled adapter may be usable for a particular sled. A base fixture 110 will preferably be equipped with an appropriate sled adapter. The sled adapter may be a bottom plate with a surface contour that maps to the surface of the sled. The sled adapter may additionally include latches or connectors that are specially designed to engage the sled attachment fixture 112 to the sled.

In some variations, the sled may include mounting features which can be used as a mounting point. The base fixture or the sled attachment fixture 112 more specifically may attach to or otherwise connect to such mounting features. The strap grooves are one example of mounting features that were previously discussed. Additional or alternative mounting features may similarly be used to connect the base fixture 110 to a sled.

Herein, the system is primarily discussed in how the base fixture 110 attaches to a sled. In an alternative variation, the base fixture 110 may mount to or attach to another nearby structure such as the ground, another portion of the scanning device, or another suitably grounded structure. In one variation, the base fixture 110 may attach to or include stand that can be positioned after the base of the sled. In this variation, the base fixture 110 may be mounted to the floor in a permanent or a temporary manner. In one example, the base fixture 110 may be a movable stand that can be rolled or otherwise moved into position.

In one variation, a base fixture 110 that rests on the floor may be a movable stand that attaches to the sled such that as the sled moves, the movable stand moves with it. In an alternative variation, the base fixture may be attached to a static location on the floor beyond the base of the sled. If the base fixture 110 remains in a stationary position, the boom support 130 may be a telescoping support member, so that as the sled moves with the in-bore fixture 120, the boom support 130 can extend.

The in-bore fixture 120 functions to establish rigid support within the bore of the scanner. The in-bore fixture 120 may be designed to establish a mounting point that is elevated above the sled such that one end of the boom support 130 can be attached to the in-bore fixture 120 at an elevated position. The elevated position can allow a subject to lie on the sled below the boom support 130. The in-bore fixture 120 may use the walls of the scanner to enable a way to further restrict motion.

In one variation, the in-bore fixture 120 is supported at one or more points of contact by the surface of the sled. In some variations, the in-bore fixture 120 rests on the sled when in use. In other variations, the in-bore fixture 120 may be more rigidly attached to the sled. In many variations, the in-bore fixture 120 is a ring-shaped structure that has two “feet” or many areas of contacting a grounded surface like the sled.

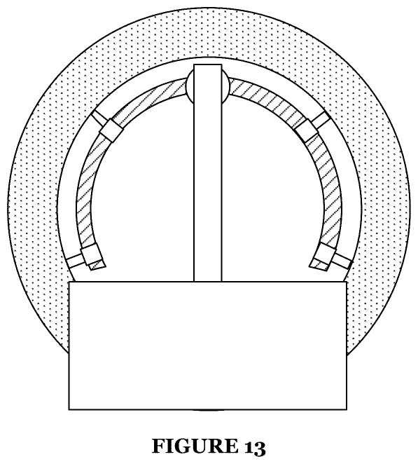

Alternatively, the in-bore fixture may be configured to potentially avoid non-bore grounded contact. In this example, the in-bore fixture 120 may primarily engage with the bore wall to restrict lateral and vertical degrees of freedom, thereby restricting movement in these directions. In particular, the adaptive borewall pressure loads supply forces that constrain position laterally and vertically within a bore of the medical scanning device. The adapative borewall pressure loads are preferably distributed across greater than 180° such that equal and opposite forces with a downward component (e.g., diagonally downward and outward) supply upward force to support the in-bore fixture as shown in FIG. 13. As a result, the in-bore fixture 120 may contact the borewall and the boom support 130 exclusively. In some variations, such a configuration may still include additional grounding contacts as supplemental supports. However, a variation avoiding additional ground contacts may enable the in-bore fixture to be fitted within a bore of a device without depending on attaching to a sled. Additionally, in some scanning device designs the motion of a sled may complicate the insertion of the in-bore fixture 120. In such cases, having an in-bore fixture 120 that can directly support itself without reliance on there being an additional non-bore ground surface/structure.

The in-bore fixture 120 may be designed to passively rest on the sled bed, which can function to make it an easily repositionable and removable object. The in-bore fixture 120, as described herein, can include design features that can make it a solid grounding fixture once positioned inside the bore. The in-bore fixture 120 could include grounding contacts 124 that function as how the in-bore fixture 120 engages with the sled or other surface. The in-bore fixture 120 is preferably a semi-circular structure. The in-bore fixture 120, in some variations additionally includes an adaptive borewall pressure load 122 that functions to supply resistive forces from the semi-circular structure to further fix the position of the in-bore fixture 120. An additional or alternative position locking mechanism may additionally be used.

The grounding contacts 124 of the in-bore fixture 120 function to establish contact with a grounding surface. The grounding surface in some variations is the sled. However, the grounding surface in some alternative variations may be part of the borewall or some alternative structure. The grounding contacts 124 in one variation contact at multiple regions. In particular, the grounding contacts 124 are oriented on either side of the width of the in-bore fixture 120 such that the grounding contacts 124 enable the in-bore fixture 120 to straddle a sled.

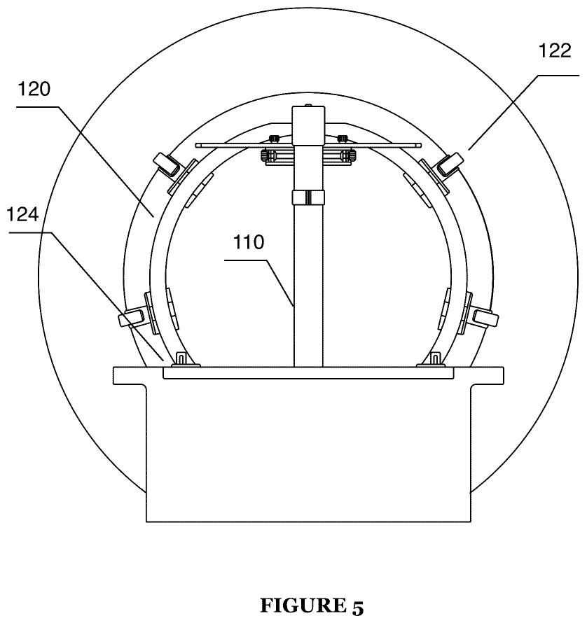

In one example, the in-bore fixture 120 may be a semi-circular structure or round partial ring with two ends of the structure serving as two feet where the in-bore fixture 120 can be supported by the sled as shown in FIG. 5. In this example, the in-bore fixture 120 may include a first grounding contact 124 positionable on one lateral side of the sled and a second grounding contact 124 on the other lateral side of the sled. In alternatively variations, there may be more than two contact regions or in some variations a single contact region. In one variation, a grounding contact 124 may extend across a significant portion of the sled. For the example, the in-bore fixture may be a semi-circle structure that has a flat plate, or profiled plate (to match sled), and/or a conformable bottom surface that extends from one side of the sled to another to increase the surface area of contact between the in-bore fixture 120's sled pad and the surface of the sled.

An in-bore fixture 120 with grounding contacts 124 may be designed for removable installation of the in-bore fixture 120 into a device. This may enable the in-bore fixture 120 to be easily removed from the sled and then added to the sled when it is ready for use. When used, an operator could, for example, set the in-bore fixture 120 at the back of the sled. The sled would be moved into the bore. An adaptive borewall pressure load 122 may then press against the bore wall such that multiple contact points between the in-bore fixture 120, the borewall of the scanner, and the sled would suitably restrict the movement of the in-bore fixture 120.

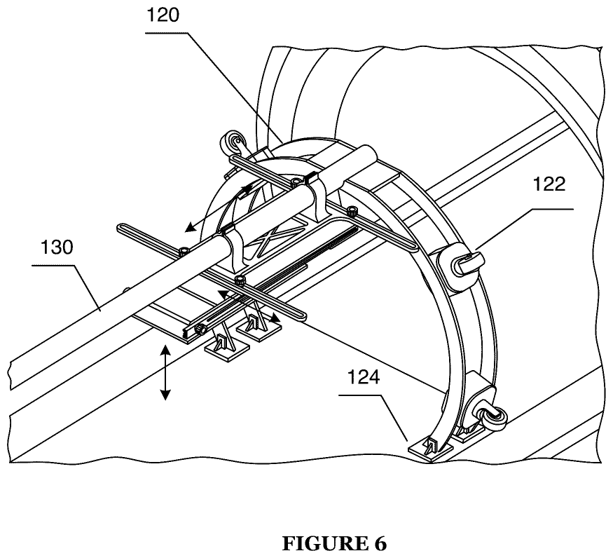

In alternative variations, in-bore fixture 120 may be attached to the sled using adhesive, attachment mechanisms, or other ways of fixing how it is supported by the sled as shown in FIG. 6.

The grounding contact 124 may be implemented in a variety of different approaches. In one variation, the grounding contacts 124 establish a friction contact with the sled. In such a variation, the grounding contact 124 is a pad or other type of contact region, which functions as a surface that can engage with the sled surface. The pad may be or configured similarly to the sled attachment fixture 112 of the base fixture 110, possibly including similar features such as a concave base, conforming base, an elevated fixture for strapping, and the like. The in-bore fixture 120, however, may additionally have loading forces from the adaptive borewall pressure load 122, which may enable additional configurations for restricting motion of the in-bore fixture 120. In one variation, the pads could be made of rubber or some other non-slip material to prevent sliding when the in-bore fixture 120 is engaged. in another variation, the pads could be adhered to the sled or more rigidly attached. A strapping approach may be applied in a similar manner to the sled attachment fixture 112.

In another variation, the grounding contact 124 may be implemented as wheeled grounding contact as shown in FIG. 2. A wheeled grounding contact may enable the in-bore fixture 120 to be more easily rolled into position. A wheeled grounding contact may include one or more wheel or rolling element. In one variation, each grounding contact 124 may include two wheels, which may function to provide enhanced stability. As shown in a variation of FIG. 2, an in-bore fixture 120 includes two wheeled grounding contacts on opposing sides of a sled. In this way the wheeled grounding contacts are oriented on either side of the width of the in-bore fixture 120 such that the grounding contacts 124 enable the in-bore fixture 120 to straddle a sled. The wheels are preferably made of non-slip materials to avoid sliding on the sled bed. In some variations, the wheels may include tracks or other features to enhance its grip on the sled surface. As an alternative to a wheel, a rolling track may be used, which may provide additional contact with the surface of the sled to prevent slippage.

In one variation, the wheeled grounding contact includes one or more spring-loaded wheels, pneumatic springs, or other suitable wheels with a restorative force. As such in some variations, the same mechanism used for the adaptive borewall pressure loads 122 may also be used as the grounding contacts. In some devices, there may be an uneven surface over which the in-bore fixture must pass over. Spring-loaded wheels attached within a wheeled grounding contact of the in-bore fixture 120 function to allow the wheels to adjust as they roll over ridges or other objects. In particular, in some MRI machines, there may be a lip or surface that the in-bore fixture 120 passes over, a spring-loaded wheels or a wheeled grounding contact with some restorative force mechanism can enable the in-bore fixture 120 to easily pass over any such obstructions.

The grounding contacts 124 may additionally include a height adjustment mechanism, which functions to allow the height setting of the grounding contacts 124 to be adjusted and by extension adjusting the height of the in-bore fixture 120.

The in-bore fixture 120 preferably has a semi-circular outer profile where a defined semi-circular perimeter fits within the scanner bore.

In particular, the in-bore fixture 120 may be or include a semi-circular structure or partial ring as shown in FIGS. 1, 2, 5, and 6. The in-bore fixture 120 generally includes a structural frame that provides the structural and geometric qualities to properly engage with the bore wall. The semi-circular partial ring may include a defined central opening, which function to allow the head of the subject (or other objects) to rest between the grounding contacts 124. In other words, a semi-circular partial ring structure forms a broken circle (e.g., a rounded “C” shape), where the two ends may be used as a base. Grounding contacts 124 may be included at the two ends of the partial ring structure. In one such variation, the semi-circular partial ring may terminate at two grounding contacts 124, which would be on opposing sides of a sled of a medical scanning device. In some alternative variations, the in-bore fixture 120 may not include such a central defined opening and could include structural elements such as a beam or plate to add structural support.

In some variations, the geometric form may not be semi-circular in shape but could have points of the structure arranged in a substantially semi-circular or a partially rounded arrangement. Herein semi-circular generally refers to a portion of the in-bore fixture 120 forming a defined circular, elliptical, or otherwise rounded path. This rounded path will generally make an arc 270°-100°. The rounded path in some variations is preferably greater than 180°, which may enable suitable opposing points of contact with the adaptive borewall pressure loads 122. The rounded path however may be any suitable degree and, in some variations, the in-bore fixture may have a full circular outer profile. The semi-circular structure may be used as a mounting surface that at least partially surrounds a subject on the sled. The semi-circular structure may additionally or alternatively be used as part of the adaptive borewall pressure load 122.

The semi-circular structure is preferably made of a rigid material. In the context of a scanning device, the in-bore fixture 120 may be made of MRI compatible material as discussed herein.

The in-bore fixture 120 may include an adaptive borewall pressure load 122, which functions to physically engage with the borewall when in a mounted state so as to restrict motion along one or more dimensions. More specifically, the in-bore fixture 120 may include adaptive borewall pressure loads 122 distributed across multiple locations on a semi-circular outer profile of the in-bore fixture. The adaptive borewall pressure loads 122 preferably direct pressure radially outward. The radially outward pressure is preferably applied from a mechanism along the outer profile.

The pressure loads are preferably adaptive in that they may be adjustable where they can be selectably engaged or modified to alter how the mechanism engages with a borewall. The pressure loads may also be adaptive in that they may be dynamic where they automatically and naturally engage with the borewall based on their design.

In some variations, there may be a set of distinct contact points that can each use some mechanism to exert an outward force against the borewall. In combination, the set of contact points can restrict motion along a defined plane perpendicular to the defined longitudinal axis. Additionally, motion in the longitudinal direction can also be restricted as a result of the outward pressure and friction between the points of contact of the in-bore fixture 120, borewall, and the sled.

The expanding force of the borewall pressure load 122 may use a spring-loaded mechanism, geared mechanism, a pneumatic spring, a ratcheted or alternatively loaded lever, an actuator, and/or any suitable mechanism to apply a force towards the borewall. The borewall pressure load may apply pressure at distinct locations or regions. In this case, the set of pressure loads preferably establishes a set of opposing forces in a two dimensional plan that is perpendicular to the longitudinal axis. This may restrict motion in the lateral and vertical axis. A pressure load that opposes the ground forces may additionally serve to reinforce positioning on the sled which can prevent slippage or movement in the longitudinal direction. Accordingly, within the in-bore fixture 120 there may be at least 3 points of contact. In many versions, the in-bore fixture includes at least two points of pressure loads that work with the two grounding contacts 124. Some alternative variations may include additional pressure loads. In yet still other variations, a mechanism used as the pressure load may apply an outward force along an extended portion of the outer profile. For example, a pressure load may be applied across a substantial portion of semi-circular structure from one mechanism.

In one variation, the adaptive borewall pressure loads 122 could be from the shape or structural design of the structure of the in-bore fixture. For example, a semi-circular partial ring structure could have integrated spring-like action such that it can be compressed inward radially when slid into a borewall and then when positioned can apply an outward radial force.

In another variation, the adaptive borewall pressure loads 122 may include a set of wheeled guides as shown in FIG. 6. The wheeled guides may function to allow enhanced movability of the in-bore fixture into and out of the bore when not engaged. Once in position, the wheeled guides can be configured to exert an outward force to restrict motion establishing a secure mounting platform. Wheeled guides can include wheels or other rolling mechanisms that are directed radially outward along the semi-circular outer profile. The wheels or rolling mechanisms can engage with the bore wall. When the sled is moving into position or if an operator is adjusting placement of the in-bore fixture 120, then the wheels or rolling mechanisms can freely roll.

The set of wheeled guides preferably includes distinct wheeled guides at different locations along the semi-circular outer profile such that outward radial pressure is applied to the borewall at different locations and in different directions. In particular, the outward radial pressure by the set of wheeled guides directs outward pressure with force vectors in both axes in a two dimensional plane (e.g., a plane defined as being perpendicular to the longitudinal axis. For example, the set of wheeled guides can include 4 or more wheels separated at distinct locations along the semi-circular outer profile, directing outward pressure with force vectors in both directions along two dimensions.

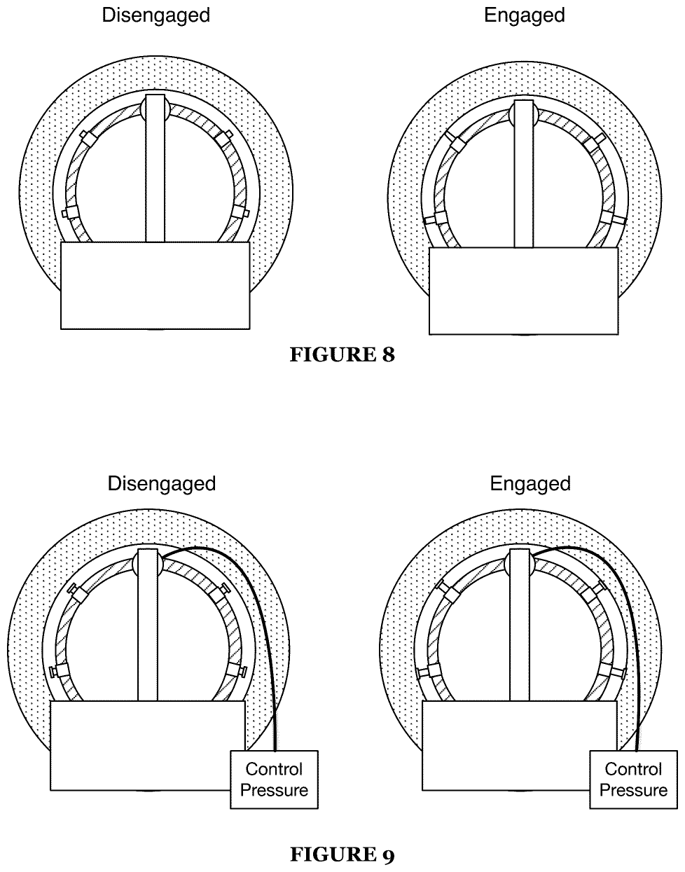

In one variation in-bore fixture includes a displacement adjustment mechanism integrated into the wheeled guides or other form of pressure loads. In one variation, the wheeled guides can include adjustable displacement. In particular the wheeled guides may include an adjustable radial displacement. The wheeled guides or pads may be adjusted to selectably engage with the borewall as shown in FIG. 8. The radial displacement can determine how far out or in the wheeled guides extend. A mechanism may be used to adjust the variable displacement. In one variation, a single control may be used to control radial displacement across all wheeled guides or a subset of wheeled guides used as pressure loads. In another variation, each wheeled guide may have individually adjustable radial displacement.

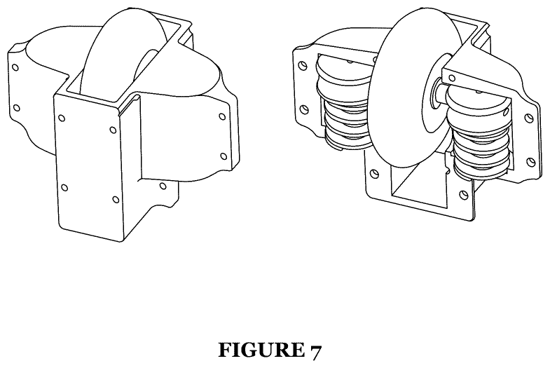

In one such wheeled variation, shown in FIG. 2, the adaptive borewall pressure load 122 can include a set of spring-loaded wheeled guides. The wheeled guides can be wheels extending outward from the structure of the in-bore fixture 120. The wheeled guides can have spring or pneumatic springs that supply a dynamic outward force as shown in FIG. 7. A spring could be a plastic spring. The wheeled guides may be adjusted so that they extend beyond the defined perimeter of the defined cylindrical cavity of the scanner's bore. When the sled is moved into the scanner, the borewall exerts a force in ward onto the wheels, thereby compressing a spring-loaded mechanism. The tension and amount of pressure may be tunable by adjusting a wheel position offset. Accordingly, in some variations, the in-bore fixture or the spring-loaded wheeled guides may have an adjustable offset. The wheels allow the in-bore fixture 120 to roll while the sled is being moved into position. The wheels may include a lock that can be engaged when the final sled position is set. Alternatively, there may be a separate locking mechanism. For example, the in-bore fixture 120 may include a brake that can be engaged to contact the bore wall and prevent further rolling. The spring-loaded wheels used as the pressure loads may additionally be used within a wheeled grounding contact.

In another variation, the adaptive borewall pressure load 122 can include a set of pressure-activated mechanisms. The pressure-activated mechanisms could pressure-activated wheeled guides, pressure-activated brake pads, or other types of pressure loaded mechanisms. Pneumatic or hydraulic pressure may be used to supply a restorative force allowing the mechanism to apply an outward force.

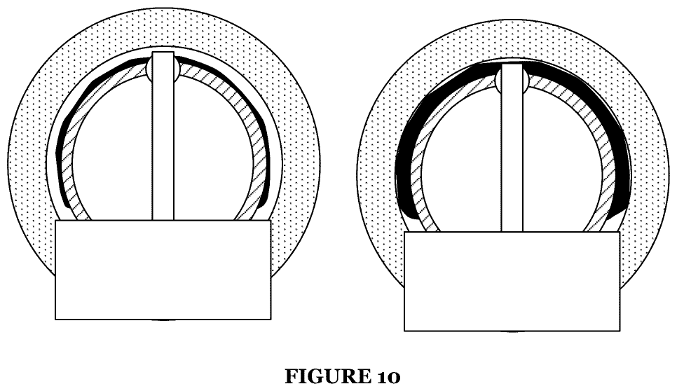

A pressure-activated mechanism may more specifically be a pneumatic or hydraulic driven mechanism. In some variations, pneumatic or hydraulic driven mechanisms may have connecting control lines supplying the pneumatic or hydraulic force as shown in FIG. 9. This may make them controllable where pressure can be deactivated to allow for easy movement in and out of the bore, and then pressurized to engage with the borewall. The pneumatic or hydraulic driven mechanisms may generally be pressurized equally and at the same time. The system can include a set of hydraulic or pneumatic control lines connected to the pressure activated wheeled guides. Changing pressure can change the engagement state of the mechanism (e.g., wheeled guides or pads). In one variation, a single channel of a pneumatic or hydraulic control lines may be connected to supply the pressure. Alternatively, multiple pneumatic or hydraulic lines may be used to control the different mechanisms individually. Activation of pressure can result in the mechanism (e.g., wheeled guide or pads) exerting an outward force. Alternatively, the pressure-activated mechanism may be inversely configured such that pressure is used to unload (deactivate) the mechanism.

In another variation, an adaptive borewall pressure load 122 may be or include an inflatable element like an inflatable tube or bladder as shown in FIG. 10. The inflatable element may be oriented at different points or along a substantial portion of the outer circumference of the in-bore fixture 120. When in position, the inflatable element may be inflated using a fluid or gas source. The inflatable element may have an outer surface made of a material that has high friction such as rubber, silicones, urethanes, or other suitable materials that can be inflatable. When deflated, the in-bore fixture has an effective outer circumference less than the bore. When inflated, the inflatable element can inflate and engage with various sizes of the bore wall. The maximum circumference of the in-bore fixture with the inflatable element inflated is preferably greater than the bore circumference.

In another variation, the adaptive borewall pressure load 122 may include a set of expanding brake pads, which can extend outwardly to engage with the borewall and then tightened. These expanding brakes could use some geared mechanism, linkage mechanism, a controllable actuator, or any suitable mechanism to move outward or inward.

In a similar variation, the in-bore fixture 120 may additionally include a brake mechanism that can be engaged. the brake mechanism may engage with the bore wall and/or the sled. In some variations, it may resist movement in the longitudinal direction through brakes may be used to fix position in any suitable manner. The brakes may be in addition or as an alternative to pressure loads.

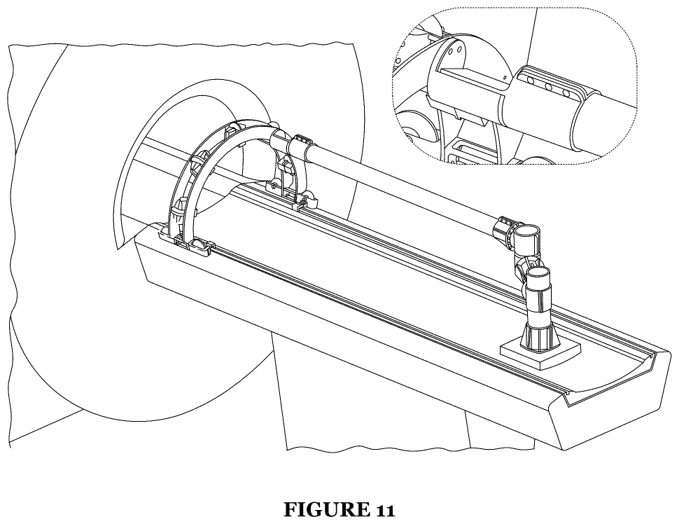

The in-bore fixture 120 may additionally include one or more attachment mechanisms that can physically couple with the boom support 130 as shown in FIG. 11. Various mechanisms or physical designs may be used. In general, in-bore fixture 120 includes at least one connection point, where one end of the boom support 130 can attach. In some variations, the in-bore fixture 120 may have the connection point actuated so that positioning is adjustable. In some variations, there may be multiple connection points, which can offer flexibility in exact positioning and/or enable a capability to have multiple boom supports 130 mounted simultaneously. The boom support 130 in some variations is selectably attachable to the in-bore fixture 120, and so the connection point may be easily engaged and disengaged from the boom support 130. This may serve to enable the boom support 130 to be disconnected and possibly moved out of the way while a patient is positioned on a sled.

The in-bore fixture 120 will generally include a singular semi-circular arc or ring that engages with the borewall. In some alternative variations, the in-bore fixture 120 may be designed with an extended longitudinal width such that it engages with more of the bore along the longitudinal direction. In one such variation, the system may include multiple instances of the in-bore fixture 120 as described above. These different in-bore fixtures instance can be physically connected as distinct in-bore fixtures or integrated into a single multi-instance in-bore fixture 120. In one variation, connecting bars or structures can extend along the semi-circular outer profile between two in-bore fixture instances. In this way multiple semi-circular rings may have adaptive pressure loads acting against the borewall. This may function to make the mounting platform of the system more secure.

While the in-bore fixture 120 is primarily described as it would be used with a base fixture 110. In some alternative variations, the system may use one or more in-bore fixtures 120 without a base fixture 110. The boom support 130 or another mounting component may be integrated directing with the in-bore fixture. In one variation, two in-bore fixture instances may have a boom support 130 suspended between them.

The boom support 130 functions as a structure that can extend from the base fixture 110 to the in-bore fixture 120. The base fixture 110 and the in-bore fixtures 120 may primarily be used in establishing rigid support, and the boom support 130 may be used to provide a mounting surface extending a partial or full length of a patient—various devices could be mounted along this mounting surface. In one variation, the boom support 130 can be a cylinder, a beam, or other suitable type of arm. Other alternative approaches could make the boom support 130 any suitable form.

In some variations, the boom support 130 is removably attached between the base fixture 110 and the in-bore fixture 120 when the system is in use. Accordingly, the boom support 130 may selectively couple or engage with the in-bore fixture 120. The system may include mechanisms on the base fixture 110, boom support 130, and/or the in-bore fixture 120 to attach and detach the boom support 130. This may be used so that when not in use or when opening up the sled for loading/unloading of a patient, the boom support 130 can be removed to provide more space around the sled.

In a similar variation, as shown in FIG. 1, the boom support 130 could be coupled to the base fixture 110, and the system could include a rotational hinge, joint, or other actuating mechanism allowing the boom arm to be rotated about the base fixture 110. This can allow the boom support 130 to be rotated or otherwise moved out of the way and then easily rotated and engaged with the in-bore fixture 120. The actuating mechanism may be integrated into the base fixture 110 and/or the boom support 130.

In other variations, the boom support 130 may be permanently or semi-permanently attached. This can be used in a variation where the mounting structure is desired to be kept in place for a sustained period of time.

In some variations, the boom support 130 may have adjustable length. The boom support 130 may include some extension element which could be a telescoping element, a slidable element, or other sort of element that can be adjusted to change the length of the boom support 130.

The boom support 130 may include one or more mounting elements. These could be mounting ports for connecting other objects. Alternatively, the boom support 130 may be adapted for use of various clamps, grips or other attachment mechanisms to engage with the boom support 130. This can enable various components to be attached to the boom support 130. While the boom support 130 is discussed as the main surface for mounting, objects may similarly be mounted to the in-bore fixture 120 and/or the base fixture 110 as required.

### 3. Mounting Fixture

The system can additionally include a mounting fixture 140, which functions to provide mounting elements that can be used to mount one or more devices. The mounting fixture 140(s) may be integrated into the boom support 130, the in-bore fixture 120, and/or any suitable component of the system.

The mounting fixtures 140 may include rails that allow mounting a device along some path, mounting holes or points, and/or other mounting mechanisms. In one exemplary implementation, the mounting fixture 140 could be a frame that mounts around the boom support 130 to enable the mounting fixture 140 to be positioned longitudinally. The mounting fixture 140 may additionally include other adjustable mounting mechanisms to provide more positioning options. For example, the mounting fixture 140 could include a set of rails to allow adjustment of a mounted objects lateral position (side-to-side). In on other variations, there may be mechanisms or ways to adjust rotational position (e.g., angle), the radial displacement, and/or other aspects.

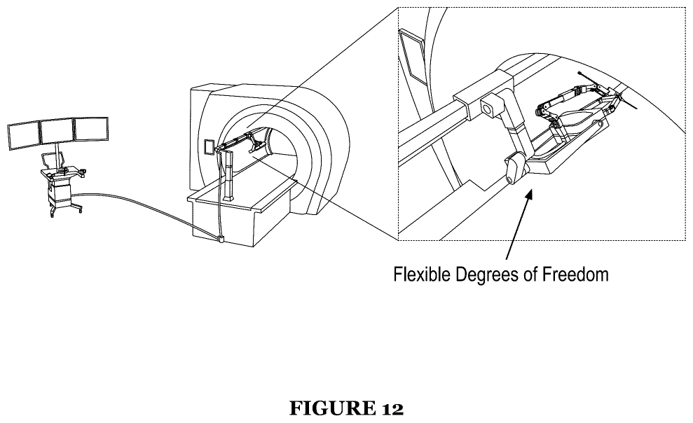

In one variation, the mounting fixture may be an actuating mechanism with multiple degrees of freedom that attaches to the boom support (or to the base fixture 110 or the in-bore fixture 120). As shown in FIG. 12, a mounting fixture that is an actuating mechanism could be a multi-segmented articulating arm. This can allow a device to flexibly be positioned around a patient.

As used herein, first, second, third, etc. are used to characterize and distinguish various elements, components, regions, layers and/or sections. These elements, components, regions, layers and/or sections should not be limited by these terms. Use of numerical terms may be used to distinguish one element, component, region, layer and/or section from another element, component, region, layer and/or section. Use of such numerical terms does not imply a sequence or order unless clearly indicated by the context. Such numerical references may be used interchangeable without departing from the teaching of the embodiments and variations herein.

As a person skilled in the art will recognize from the previous detailed description and from the figures and claims, modifications and changes can be made to the embodiments of the invention without departing from the scope of this invention as defined in the following claims.

We claim:

1. A system for use in a medical scanning device comprising:
a base fixture;
an in-bore fixture with a semi-circular outer profile, and which comprises adaptive borewall pressure loads distributed across multiple locations on the outer profile, wherein the adaptive borewall pressure loads direct pressure radially outward; and
a boom support attachable between the base fixture and the in-bore fixture.

2. The system of claim 1, wherein the in-bore fixture is a semi-circular partial ring that terminates at two grounding contacts on opposing sides of a sled of the medical scanning device.

3. The system of claim 1, wherein the adaptive borewall pressure loads supply forces constraining position laterally and vertically within a bore of the medical scanning device.

4. The system of claim 1, wherein the adaptive borewall pressure loads are a set of wheeled guides.

5. The system of claim 4, wherein the set of wheeled guides comprises wheels separated at distinct locations of the semi-circular outer profile, directing outward pressure with force vectors in both directions along two dimensions.

6. The system of claim 4, wherein the set of wheeled guides are spring-loaded wheeled guides.

7. The system of claim 4, wherein the set of wheeled guides are pressure-activated wheeled guides.

8. The system of claim 7, further comprising a set of hydraulic or pneumatic control lines connected to the pressure activated wheeled guides.

9. The system of claim 4, wherein the wheeled guides each include an adjustable radial displacement.

10. The system of claim 1, wherein the in-bore fixture comprises grounding contacts on opposing sides of a sled of the medical scanning device, the grounding contacts establishing friction contact with a sled of the medical scanning device.

11. The system of claim 1, wherein the in-bore fixture comprises wheeled grounding contacts on opposing sides of a sled of the medical scanning device.

12. The system of claim 1, wherein the base fixture comprises a rotational joint and a boom attachment mechanism to selectively couple the boom support to the in-bore fixture.

13. The system of claim 1, wherein the base fixture comprises a sled attachment fixture that is strapped to a sled of the device.

14. The system of claim 13, wherein the sled attachment fixture comprises a structural element that extends vertically to a height above where a strap attaches to a sled.

15. The system of claim 14, wherein the sled attachment fixture comprises a conformant bottom surface.

16. The system of claim 15, wherein the conformant bottom surface is a convex bottom surface.

17. The system of claim 1, wherein the base fixture comprises a bottom surface that is adhered to a surface of a sled of the medical scanning device.

18. The system of claim 1, wherein the base fixture is rigidly attached to a sled of the medical scanning device semi-permanently.

19. The system of claim 1, further comprising a sled attachment fixture and an attachment connector, wherein the attachment connector physically couples the sled attachment fixture to a sled of the medical scanning device.

20. The system of claim 1, further comprising a mounting fixture attached to the boom support, wherein the mounting fixture actuates with multiple degrees of freedom.

21. The system of claim 1, wherein the base fixture, in-bore fixture, and the boom support are made of materials compatible with magnetic resonance imaging devices.
